(19) United States
(12) Patent Application Publication
(10) Pub. No.: US 2005/0267688 A1
(43) Pub. Date: Dec. 1, 2005

(54) Method for enhanced accuracy in predicting peptides elution time using liquid separations or chromatography

(76) Inventors: Konstantinos Petritis, Richland, WA (US); Lars J. Kangas, West Richland, WA (US); Gordon A. Anderson, Benton City, WA (US); Richard D. Smith, Richland, WA (US)

Correspondence Address: Douglas E. McKinley, Jr.;McKinley Law Office, P.O. Box 202, Richland, WA, 99352, US

(21) Appl. No.: 10/846,188

(22) Filed: May 14, 2004

Related U.S. Application Data
(63) Continuation-in-part of application No. 10/323,387, filed on Dec. 18, 2002.

(51) Int. Cl. G06F 19/00; G01N 33/48; G01N 33/50

(52) U.S. Cl. 702/19

(57) ABSTRACT

A method for predicting the elution time of a peptide in chromatographic and electrophoretic separations by first providing a data set of known elution times of known peptides, then creating a plurality of vectors, each vector having a plurality of dimensions, and each dimension representing positional information about at least a portion of the amino acids present in the known peptides. A hypothetical vector is then created by assigning dimensional values for at least one hypothetical peptide, and a predicted elution time for the hypothetical vector is created by performing at least one multivariate regression fitting the hypothetical peptide to the plurality of vectors. Preferably, the multivariate regression is accomplished by the use of an artificial neural network and the elution times are first normalized using linear regression.

### CROSS-REFERENCE TO RELATED APPLICATIONS

This application is a Continuation in Part of U.S. patent application Ser. No. 10/323,387, filed Dec. 18, 2002, the entire contents of which are incorporated herein by this reference.

### STATEMENT REGARDING FEDERALLY SPONSORED RESEARCH OR DEVELOPMENT

This invention was made with Government support under Contract DE-AC0676RLO1830 awarded by the U.S. Department of Energy. The Government has certain rights in the invention.

### REFERENCE TO SEQUENCE LISTING

Each protein sequence described herein has been submitted to the U.S. Patent and Trademark Office on a compact disc in computer readable form in compliance with 37 CFR §§ 1.821-1.825. A paper copy of that submission is attached herewith. The sequence listing information recorded in computer readable form is identical to the written sequence listing.

### BACKGROUND OF THE INVENTION

Liquid phase separations (eg. liquid chromatography and electrophoretic separations) have long been used as investigative tools by scientists and researchers seeking to identify the structure of molecules, particularly peptides (as used herein the term “peptides” refers to polymers having more than one amino acid, and includes, without limitation, dipeptides, tripeptides, oligopeptides, and polypeptides. The term “protein” refers to molecules containing one or more polypeptide chains).

Proteomics involves the broad and systematic analysis of proteins, which includes their identification, quantification, and ultimately the attribution of one or more biological functions. Proteomic analyses are challenging due to the high complexity and dynamic range of protein abundances. The industrialisation of biology requires that the systematic analysis of expressed proteins be conducted in a high-throughput manner and with high sensitivity, further increasing the challenge. Recent technological advances in instrumentation, bio-informatics and automation have contributed to progress towards this goal. Specifically, in the area of proteomic identification, it is evident that greater specificity benefits the ability to deal with the high complexity of proteomes. As a result, recent efforts have focused on improvements in separation speed, resolving power and dynamic range, and these methods have generally been based on the combination of separations with mass spectrometry (MS), using correlation of tandem mass spectra with established protein databases or predictions from genome sequence data for identifications.

Additionally, modern proteomics research has increasingly taken advantage of the ability of liquid chromatography to identify proteins from their elution time from a chromatographic column. The information gleaned from a liquid chromatograph can be enhanced by identifying the molecule's mass, or mass to charge, by coupling the liquid chromatograph either on line or off line, with a mass spectrometer. Common methods include offline tryptic digestion and subsequent electrophoretic or chromatographic separation with matrix-assisted laser desorption/ionization or electrospray time-of-flight or ion trap mass spectrometry. Capillary electrophoresis, mass spectrometry or liquid chromatography/mass spectrometry coupled online via electrospray interfaces have also been used to analyze tryptic and other digests of complex biological samples such as whole cell lysates and human body fluids. The dynamic range of the mass spectrometer in these methods may be limited when a sample is directly infused by ion suppression in the electrospray and the detector. Further, the dynamic range of Fourier transform ion cyclotron resonance (FTICR) and ion trap mass spectrometers can be limited by the storage capacity within the instrument, although it has been shown that the use of a mass selective quadrupole to selectively load the FTICR cell.

Researchers attempting to enhance the accuracy of these methods have devised a number of schemes to increase their accuracy. For example, in the paper “Prediction of Chromatographic Retention and Protein Identification in Liquid Chromatography/Mass Spectrometry” Magnus Palmblad, Margareta Ramstrom, Karin E. Markides, Per Hakansson, and Jonas Bergquist, Analytic Chemistry p. 4-9, 2002, the authors describe a method for using the information from liquid separation schemes such as chromatography and electrophoretic methods, to improve peptide mass fingerprinting based on accurate mass measurement. The author's concede that the resolving power and accuracy in chromatographic separations are several orders of magnitude lower than in mass spectrometry, but they contend that the information is complementary in nature and available at negligible computational cost and at no additional experimental cost. Briefly, the method described in the Palmblad paper assigns “retention coefficients” for the 20 amino acids, as well as the number of each amino acid, a term that compensates for void volumes and a delay between sample injection and acquisition of mass spectra. The parameters are then fitted by the least squares method to experimental data from ˜70 BSA peptides of ˜100 HAS and transferrin peptides putatively identified by accurate mass measurement and high relative intensities in the mass spectra. The authors found that “the accuracy of the predictor was found to be 8-10% when “trained” by each of the six BSA and CSF data sets.” While approaches such as that described in the Palmblad paper provide some useful information, their utility is limited by the accuracy of the predictions.

Thus, at the present, there are two major approaches for proteomic analyses. The first one consists of the off-line combination of two-dimensional polyacrylamide electrophoresis (2D-PAGE) with MS. The proteins are first separated in a gel by their pI and mass and then the protein “spots” are enzymatically hydrolysed resulting in peptide mixtures which are analysed by matrix assisted laser desorption ionisation-time of flight (MALDI-TOF) or electrospray (ESI)-MS. Another rapid evolving approach consists of a global proteome-wide enzymatic digestion followed by analysis using on-line 1-D or 2-D liquid chromatography (LC) coupled with ESI-MS. The detection of the peptides is achieved by tandem MS or more recently by single stage Fourier transform ion cyclotron resonance (FTICR)-MS, which provides high sensitivity, large dynamic range and high throughput in routine applications by circumventing the need for tandem MS.

An aspect of proteomic analysis that has not yet been exploited involves use of the information available from the separations (eg. LC elution time). Indeed, retention time in LC is unique and structurally dependent for a defined experiment (mobile phase composition, stationary phase etc.). If there is a way to predict the LC retention time for a given peptide structure, then this could be used in conjunction with either MS/MS data to improve the confidence of peptide identifications and/or increase the number of peptide identifications, or, with sufficiently high accuracy MS, to reduce the need for MS/MS data (i.e. if the prediction is reliable enough).

The idea that chromatographic behaviour of peptides could be predicted based on the amino acid composition is not new. In 1951, Knight and Pardee showed that synthetic peptides retention factor (R_{f}) values on paper chromatography could be predicted with some accuracy. In 1952, Sanger introduced the problem of isomers by demonstrating that the relationship between R_{f} and composition was not absolutely accurate since peptides containing the same amino acids but having difference sequences could frequently be separated. More recently, there have been several reports on the prediction of peptide elution times in reversed-phase (RP) or normal phase liquid chromatography. These methods used quantitative structure-chromatographic retention relationships (QSRR's) (e.g. partial least square or multiple linear regression) for the peptide elution time prediction. Casal et al. demonstrated that partial least squares regression provides a better predictive ability with these models using a mixture of 25 small standard peptides. One limitation of these models is that they are most effective for peptides with less than 15-20 amino acid residues.

Another approach, based on artificial neural networks (ANNs), has demonstrated better predictive capabilities in several areas of chemistry including: (i) conformational states for small peptides, (ii) carbon-13 nuclear magnetic resonance chemical shifts and (iii) the retardation factor or retention time of small molecules in thin layer chromatography, GC and LC. One of the reasons is that a large number of empirical observations are needed in order to generate a sufficient populated training set for the artificial neural network. These numbers could only be achieved after the introduction of LC-MS and special statistical tools which provide automated spectra interpretation like the commercially available program “SEQUEST”.

In U.S. patent application Ser. No. 10/323,387, filed Dec. 18, 2002, the inventors of the present invention describe a method for predicting the elution or retention times of chemically related compounds such as proteins and peptides in liquid separations. (For convenience, this disclosure will hereafter refer to both proteins and peptides simply as <<peptides >>, with the understanding that the use of the term peptides is intended to encompass any biomolecule containing two or more amino acids.) Briefly, the method begins by first providing a data set of known elution times of known peptides. This data is typically taken from multiple separation experiments. A plurality of vectors is then created, each vector having a plurality of dimensions, and each dimension representing the elution time of amino acids present in each of these known peptides from-the data set. The elution time of any peptides may then be predicted by first creating a vector by assigning dimensional values for the elution time of amino acids of at least one hypothetical peptide and then calculating a predicted elution time for the vector by performing a multivariate regression of the dimensional values of the hypothetical peptide using the dimensional values of the known peptides. Preferably, the multivariate regression is accomplished by the use of an artificial neural network (hereinafter referred to as an “ANN”), such as a “feed forward” ANN. Training the ANN may be accomplished by gradient descent algorithms, such as a backpropagation algorithm or a quickprop algorithm, or by conjugate gradient algorithms. Prior to the assignment of the vectors assigned to each of the known peptides in the data set and the dimensional values of the hypothetical peptide, the elution times of the multiple separation experiments used to generate the data set are normalized using a linear or non-linear function, which may be optimized by performing multiple regressions. While the advances taught and described in U.S. patent application Ser. No. 10/323,387 has shown increased accuracy when compared with other prior art methods, there remains a need for methods for predicting the identity of peptides and proteins with even greater accuracy.

### BRIEF SUMMARY OF THE INVENTION

Accordingly, it is an object of the present invention to provide a method for predicting the elution or retention times of chemically related compounds such as proteins and peptide in liquid separations. As used herein, “liquid separations” includes, but is not limited to, different modes of liquid chromatography,(i.e. normal and reverse phase, ion-exchange, hydrophophilic interaction chromatography, size exclusion, hydrophobic chromatography, etc) electrophoretic separations, such as capillary electrophoresis; gas chromatography, ion-mobility, field flow fractionation, and methods whereby one or more of these techniques are combined. Furthermore it can be applied in the analytical or preparative mode of the above methods. These and other objects of the present invention are accomplished- by enhancing the method taught in U.S. patent application Ser. No. 10/323,387 (hereinafter the referred to as the “prior method”) by incorporating additional information into the prior method. Specifically, the present invention makes use of the fact that the elution times of various peptides are affected not only by the total number of each of the amino acids present in a peptide, but also by the order of the amino acids in the peptide. The improved method thus begins in the same manner as the prior method, by first providing a data set of known elution times of known peptides. This data is typically taken from multiple separation experiments. In one embodiment of the present invention, as in the prior method, a plurality of vectors is then created with each vector having 20 dimensions corresponding to each of the 20 amino acids, and each dimension thus representing the elution time of the specific amino acids present in each of these known peptides from the data set. However, in this embodiment of the present invention, the amino acids present at the beginning and end of the peptide are excluded from this vector. The vector thus consists of 20 dimensions, with each dimension represented by the number of times a given amino acid appears in the middle of each peptide.

This embodiment of the present invention improves on the prior method by then providing another group of vectors that incorporate positional information about amino acids at the beginning and end of the known peptides that was previously excluded. By way of example, and not meant to be limiting, this positional information might include vectors for the first and last eight positions along a peptide. Continuing the example, each positional vector would have 20 dimensions (one for each possible amino acid). For the first position, whichever amino acid were present in the first position of the peptide would be represented by a “1”, and all remaining dimensions in the vector would be represented by zeros. A vector would then be created for each of the remaining positions. Thus, in this example, 340 total dimensions are possible; 8 positions at the beginning of the peptide multiplied by 20 possible amino acids, added to 8 positions at the end of the peptide also multiplied by 20 possible amino acids and finally an additional 20 dimensions, with each dimension representing the number of times each amino acid appears in the middle of each peptide. The vectors are thus correlated to the elution times for any peptide having the same combination of amino acids, with enhanced accuracy provided by the positional data provided for the first and last 8 amino acids.

The above description and examples have assumed that the peptides being identified by the present invention contain only 20 proteogenic amino acids (Asp, Asn, Gly, Val, Leu, Ile, Met, Phe, Trp, Pro, Ser, Thr, Cys, Tyr, Gln, Ala, Glu, Lys, Arg, His). Peptides containing other than the 20 proteogenic amino acids can be predicted accurately using the present invention assuming enough data to train the artificial neural network (i.e. retention time information of several peptides containing that amino modified amino acid). As will be recognized by those having skill in the art having the benefit of this disclosure, additional amino acids can easily be integrated into the present invention. For example, modifications might come from natural or biological processes (i.e. a protein has been phosphorilated to a Ser due to a post-translational modification) or otherwise can be artificially modified through a derivatization procedure (i.e. a protein has been reduced and alkylated at the cysteins). Under these conditions, the vectors described herein are simply expanded to account for the additional amino acids presented by such possibilities.

The elution time of any protein may thus be predicted by combining the information from the prior method with the positional information as taught herein. By first creating a vector by assigning dimensional values for the elution time of amino acids of at least one hypothetical peptide, combined with the dimensional values for the elution times for the positional information for the hypothetical peptide, a predicted elution time may be calculated for the vector by performing a multivariate regression of the dimensional values of the hypothetical peptide using the dimensional values of the known peptides.

As will be recognized by those having skill in the art having the benefit of this disclosure, the dimensional values of the prior method need only be calculated for those amino acids for which the positional information is not used. Thus, continuing with the prior example, to predict a peptide having 50 amino acids, the first and last 8 amino acids would be accounted for using the positional information (for a total of 16), and the 34 amino acids in the middle of the peptide (50 minus 16) would be accounted for using the prior method. As will further be recognized by those having skill in the art having the benefit of this disclosure, by using more than 8 amino acids at the beginning and end of the peptide, it is possible that the necessity of using any of the information from the prior method could be eliminated entirely. While a preferred embodiment of the present invention, described below, has been shown to produce the greatest accuracy by using only 16 amino acids; 8 at the beginning and 8 at the end of the peptide, this is not the result of a limitation of the present invention to the use of the positional information of only 16 amino acids. Rather, it is a limitation of the size of the data set used to train the artificial neural network used in the preferred embodiment. As new peptides are continuously being added to the data set, the data set is continually expanding. Thus, when using the method of the present invention, the optimal number of amino acids that are used in vectors created using the positional information will also continue to expand as the data set expands, and the number of amino acids that are represented using the prior method will continue to shrink. Thus, assuming, by way of example, that the universe of peptides that are of interest is limited to peptides having 50 or fewer amino acids, the database will eventually expand such that the most accurate predictions will be made by creating vectors for the first and last 25 positions of the amino acids. At that point, it will no longer be necessary to utilize any of the information for the amino acids in the middle of the peptide using the prior method, as all of those amino acids will be accounted for using the new method. Thus, while one embodiment of the new method described herein utilizes only the first and last 8 amino acids in the positional vectors, and the prior method for the amino acids in between, as the database expands, the number of amino acids used in the positional vectors will likewise expand to the point that the use of the vector created by the prior method is no longer preferred. Accordingly, those having ordinary skill in the art and the benefit of this disclosure will be able to easily adjust the number of amino acids accounted for by the positional vectors to produce the optimum results when utilizing expanded data sets, and the use of any such number of amino acids accounted for using the positional vectors are explicitly contemplated by this disclosure.

In furtherance of fulfilling their duty to disclose the best method of practicing the method of the present invention known by the applicant's herein, the applicants expect that as databases of peptides utilized by the present invention expand, the optimal number of amino acids specified by their positional information will likewise expand. Thus, another embodiment explicitly disclosed herein contemplates the use of the positional information for all of the amino acids, eliminating the need to use the prior method to account for the amino acids in the middle of the peptide.

In addition to the positional information, additional vectors can also be added to enhance the accuracy of the predictive power of the present method. For example, vectors for the peptide length, nearest neighbor effect, hydrophobic moment, hydrophobicity, peptide mass, molecular volume, quasi sequence order, secondary structure, and combinations thereof can also be combined with the above described vectors for the positional information and/or the middle section of the peptide. It is important to note that these types of additional vectors have particular utility in enhancing the accuracy of predictions when using relatively small data sets. As larger data sets are used, this information may become less advantageous, and may in some instances actually degrade the accuracy of predictions.

Thus, in one embodiment the present invention makes use of vectors made up from the positional information of the first and last amino acids in a peptide. As with the prior method, these vectors are then utilized to provide a method for predicting the elution time of chemically related compounds in liquid separations. The method thus begins by providing a data set of known elution times of known peptides, then creating a plurality of vectors, each vector having a plurality of dimensions, and each dimension representing positional information about at least a portion of the amino acids present in the known peptides. A hypothetical vector is then created by assigning dimensional values for at least one hypothetical peptide, and a predicted elution time for the hypothetical vector is created by performing at least one multivariate regression fitting the hypothetical peptide to the plurality of vectors. The present invention may further make use of vectors made up of quantitative information from the interior amino acids of the peptide as in the prior method, if the positional information has not fully accounted for all of the amino acids present in a particular peptide, and it may make use of vectors that contain information about other physical attributes of the peptide, including, but not limited to, peptide length, nearest neighbor effect, hydrophobic moment, hydrophobicity, peptide mass, molecular volume, quasi sequence order, secondary structure, and combinations thereof.

Preferably, the multivariate regression is accomplished by the use of an artificial neural network (hereinafter referred to as an “ANN”), and more preferably, the ANN is a “feed forward” ANN. Training the ANN may be accomplished by any of the training methods known in the art, including, but not limited to gradient descent algorithms and conjugate gradient algorithms. Preferred gradient descent algorithms include, but are not limited to a backpropagation algorithm and a quickprop algorithm. Prior to the assignment of the vectors assigned to each of the known peptides in the data set and the dimensional values of the hypothetical peptide, it is preferable to normalize the elution times of the multiple separation experiments used to generate the data set using a linear or non-linear function. It is further preferred to optimize this function by performing multiple regressions. The preferred method for the multiple regressions is a genetic algorithm.

The operation and use of the method of the present invention is described in a detailed description of a preferred embodiment of the present invention below. Those having skill in the art will readily recognize equivalent methods exist for the particular algorithms selected for the multivariate regression, the transfer function, and the method used to train the ANN in this preferred embodiment. Similarly, while the preferred embodiment describes the method of the present invention as it was applied in a liquid chromatograph coupled with a mass spectrometer, those having skill in the art will recognize that the method of the present invention is applicable with or without the use of the mass spectrometer, and the data provided by the mass spectrometer. Further, those having skill in the art will similarly recognize that the benefits provided by the present invention are also applicable if the mass spectrometer is replaced with other suitable detection means. It will also be apparent that while the preferred embodiment describes the method of the present invention in conjunction with liquid chromatography, the present invention should be understood to include both all the different modes of chromatography (i.e. normal phase, reversed phase, ion-exchange etc.), and further may readily be utilized with other separation techniques, including without limitation, electrophoretic separations. Accordingly, it will be apparent to those skilled in the art that many changes and modifications may be made from the preferred embodiment described herein without departing from the invention in its broader aspects, and all separation methodologies, whether used with or without a detection means such as a mass spectrometer, and all equivalent algorithms for the multivariate regression, transfer functions, and methods used to train an ANN should be interpreted as falling within the true spirit and scope of the invention as set forth in the appended claims.

### BRIEF DESCRIPTION OF THE SEVERAL VIEWS OF THE DRAWING

FIG. 1 is a schematic representation of a first preferred embodiment of the artificial neural network architecture utilized in the present invention showing 342 input nodes, 6 hidden nodes and 1 output node (342-6-1).

FIG. 2 is a schematic representation of a second preferred embodiment of the artificial neural network architecture utilized in the present invention showing wherein all of the positions of all the amino acid residues are specified in each peptide. As shown in the figure, this architecture contains 1000 input nodes, hidden nodes are still unspecified, and contains one output node.

FIG. 3 is a diagram showing the predicted vs. observed normalised elution time correlation of peptide elution time prediction model previously published by Meek, J. L. Proc. Natl. Acad. Sci. U.S.A. 1980, 77, 1632-1636), the entire contents of which are incorporated herein by this reference.

FIG. 4 is a diagram showing the predicted vs. observed normalised elution time correlation obtained with the method described in U.S. patent application Ser. No. 10/323,387, filed Dec. 18, 2002.

FIG. 5 is a diagram showing the predicted vs. observed normalised elution time correlation obtained utilizing a preferred embodiment of the present invention having an ANN architecture of 342 input nodes, 6 hidden nodes and 1 output node (342-6- 1).

FIG. 6 is a diagram showing the prediction error distribution of a peptide elution time prediction model previously published as Meek, J. L. Proc. Natl. Acad. Sci. U.S.A. 1980, 77, 1632-1636). As shown in the figure, 95% of the peptides are eluted within ±12.2% while 50% of the peptides are eluted within ±3.27%.

FIG. 7 is a diagram showing the prediction error distribution of the method described in U.S. patent application Ser. No. 10/323,387, filed Dec. 18, 2002. As shown in the figure, 95% of the peptides are eluted within ±11.15% while 50% of the peptides are eluted within ±2.56%.

FIG. 8 is a diagram showing the prediction error distribution utilizing a preferred embodiment of the present invention having an ANN architecture of 342 input nodes, 6 hidden nodes and 1 output node (342-6-1). As shown in the figure, 95% of the peptides are eluted within ±6.8% while 50% of the peptides are eluted within ±1.5%.

### DETAILED DESCRIPTION OF A PREFERRED EMBODIMENT OF THE INVENTION

A series of experiments were undertaken to demonstrate the ability of a preferred embodiment of the present invention to provide superior prediction of the elution time of peptides when compared with prior art methods. Protein was exctracted from several species of bacteria using a common preparation procedure as follows. The bacteria cells were cultured in TGY medium to an approximate 6000D of 1.2 and harvested by centrifugation at 10,000 g at 4° C. Prior to lysis, cells were resuspended and washed three times with 100 mM ammonium bicarbonate and 5 mM EDTA (pH 8.4). Cells were lysed by beating with 0.1-mm acid zirconium beads for three 1-min cycles at 5000 rpm. The samples were incubated on ice for 5 min between each cycle of bead beating. The supernatant containing soluble cytosolic proteins was recovered after centrifugation at 15,000 g for 15 min to remove cell debris. Proteins were denatured and reduced by addition of guanidine hydrochloride (6 M) and DTT (1 mM), respectively, followed by boiling for 5 min. Prior to digestion, samples were desalted using a 5000 molecular weight cut-off “D-salt” gravity column (Pierce, Rockford, Ill.) equilibrated in 100 mM ammonium bicarbonate (pH 8.4). Proteins were enzymatically digested at an enzyme/protein ration of 1:50 (w/w) using sequencing grade modified trypsin (Promega, Madison, Wis.) at 37° C. for 16 h.

Protein was then extracted from human mammary epithelial cells (HMEC) using a common preparation procedure as follows. Cell pellets were washed three times in 1 mL ice-cold phosphate buffered saline (PBS), pH 7.2, followed by centrifugation at 10,000 ×g. Lysis buffer (10 mM sodium phosphate, pH 7, 0.5% sodium dodecyl sulfate) was added to the cell pellets and the cells were lysed using sonication on ice for 5 min. The lysate was centrifuged for 15 min at 4° C., 14,000×g to pellet any cell debris. The lysate sample was denatured thermally (100° C. for 5 min) and reduced with 10 mM fresh DL-dithiothreitol (DTT, Boehringer Mannheim, Indianapolis, Ind., USA) for 1 h at room temperature (RT), followed by separation and alkylation of one aliquot with 32 mM iodoacetamide for 1 h at RT. Excess alkylation material was quenched by the addition of fresh 10 mM DTT to the samples (with incubation for 1 h at RT). Sequencing grade, modified porcine trypsin (Promega, Madison, Wis., USA) was added at a trypsin:protein ratio of 1:50 and incubated at 37° C. for 16 h, after which the samples were lyophilized to dryness and stored frozen at −80° C.

HPLC-grade water and acetonitrile were purchased from Aldrich (Milwaukee, Wis.). Fused-silica capillary columns (30-60 cm, 150 μm i.d.×360 μm o.d., Polymicro Technologies, Phoenix, Ariz.) were then packed with 5-μm C18 particles as described in Shen, Y.; Zhao, R.; Belov, M. E.; Conrads, T. P.; Anderson, G. A.; Tang, K.; Pasa-Tolic L.; Veenstra, T. D.; Lipton, M. S.; Udseth, H. R.; Smith, R. D.; Anal. Chem. 2001, 73, 1766-1775, the entire contents of which are hereby incorporated herein by this reference. Briefly, capillary RPLC was performed using an ISCO LC system (model 100DM, ISCO, Lincoln, Nebr.). The mobile phases for gradient elution were (A) acetic acid/TFA/water (0.2:0.05:100 v/v) and (B) TFA/acetonitrile/water (0.1:90:10, v/v). The mobile phases, delivered at 5000 psi using two ISCO pumps, were mixed in a stainless steel mixer (˜2.8 mL) with a magnetic stirrer before flow splitting and entering the separation capillary. Fused-silica capillary flow splitters (30-mm i.d. with various lengths) were used to manipulate the gradient speed. Capillary RPLC was coupled on-line with MS through an ESI interface (a stainless steel union was used to connect an ESI emitter and the capillary separation column). The peptide database has been generated by using several mass spectrometers including 3.5, 7, and 11.4 telsa FTICR instruments (described in detail in Harkewicz, R.; Belov, M. E.; Anderson, G. A.; Pa\check{s}a-Tolić, L.; Masselon, C. D.; Prior, D. C.; Udseth, H. R.; Smith, R. D.; J. Am. Soc. Mass Spectrom. 2002, 13, 144-154, and references therein, the entire contents of which are hereby incorporated by this reference), as well as several ion-trap mass spectrometers (LCQ, LCQ Duo, LCQ DecaXP; ThermoFinnigan, San Jose, Calif.). The ANN software used was NeuroWindows version 4.5 (Ward Systems Group, USA) and utilized a standard backpropagation algorithm on a Pentium 1.5 GHz personal computer.

Nearest-neighbor effect The simplest and direct way to incorporate the nearest-neighbor effect is to construct a 20×20 dimensional array which includes all 400 possible combinations: AA, AC, AD and et. al., and then to count the number of these bipeptides in given peptide. However the resulted data will be very sparse since a large amount of array elements is zero (the average length of tryptic digested peptides is 17±9 in the study). To avoid this bad case, the nearest-neighbor list was alternately constructed based on the amino acid property. Traditionally, 20 amino acids can be divided into 5 groups based on their side chains properties: nonpolar aliphatic (AGILPV), polar uncharged (CMNQST), aromatic (FWY), positively charged (HKR) and negatively charged (DE) groups. This division is also consistent with contribution of individual amino acid in peptide retention time prediction shown in table 2 of the reference Petritis, K., Lars, J. K., Ferguson, P. L. et al. Use of artificial neural networks for the accurate prediction of peptide liquid chromatography elution times in proteome analyses. Anal. Chem. 2003, 75:1039-48, the entire contents of which are incorporated herein by this reference. Thus we constructed a largely reduced dense 5×5 dimensional nearest-neighbor list.

Quasi-sequence-order approach Duo to the huge number of possible sequence order patterns, it is hard to directly incorporate the sequence order effect into a statistical prediction algorithm. An approximate method, called “quasi-sequence-order” approach, first introduced in the publication Chou, K. C. Prediction of protein subcellualr locations by incorporating quasi-sequence-order effect. Biochem. and Biophys. Res. Commun. 2000, 278:477-83, Chou, K. C. Prediction of protein cellular attributes using pseudo-amino acid composition. Proteins: Struct. Funct. Genet. 2001, 43:246-55, the entire contents of which are incorporated herein by reference, was used and showed successful prediction of protein sub-cellular locations and attributes. The idea was to assume that the sequence order effect of L amino acids which consisting of a₁a₂a₃a₄a₅ . . . a_{L}, can be approximately reflected through a set of sequence-order-coupling factors as defined below:

$$\begin{matrix}{τ}_{1} = \frac{1}{L - 1} {\sum }_{i = 1}^{L - 1} {J}_{i , i + 1} \\ {τ}_{2} = \frac{1}{L - 2} {\sum }_{i = 1}^{L - 2} {J}_{i , i + 2} \\ {τ}_{3} = \frac{1}{L - 3} {\sum }_{i = 1}^{L - 3} {J}_{i , i + 3} \\ ⋮ \\ {τ}_{λ} = \frac{1}{L - λ} {\sum }_{i = 1}^{L - λ} {J}_{i , i + λ} , ( λ < L )\end{matrix} \qquad ( 1 )$$

where τ₁ denotes the 1^{st}-rank sequence-order coupling factor that reflects the sequence order correlation between all the most contiguous residues along a peptide sequence, τ₂ is the 2^{nd}-rank sequence-order-coupling factor that reflects the sequence order correlation between all the second most contiguous residues, and so forth. For some special purposes at which λ≧L, we assign τ_{λ}=0. The correlation function is given by
J_{i,j}=D²(aᵢ,aⱼ)
where D(aᵢ,aⱼ) is the physicochemical evolution distance from amino acid aᵢ to amino acid aⱼ that was derived based on the residue properties hydrophobicity, hydrophilicity, polarity and side-chain volume as shown in Table 1 of Schneider, G. and Wrede, P. The rational design of amino acid sequences by artificial neural networks and simulated molecular evolution: de novo design of an idealized leader peptidase cleavage site. Biophys. J. 1994, 66:335-44, the entire contents of which are incorporated herein by this reference.

Secondary structural contents To incorporate the conformational effect, the predicted secondary structural contents (SSC, percentage of residues in the respective secondary structural states α-helix, β-sheet and coil) of a given peptide to was introduced to quantify this conformational information. The SSC was predicted relying only on the knowledge of the amino acid composition where the shared program SSCP was applied as shown in the publication Eisenhaber, F.; Imperiale, F.; Argos, P. and Frommel, C. Prediction of secondary structural content of proteins from their amino acid composition along. I. New analytic vector decomposition methods. Proteins: Struct. Funct. Genet. 1996, 25:157-68, the entire contents of which are incorporated herein by this reference. Generally only peptides with adequate length have secondary structure, therefore the SSP was employed only when the peptide length was not smaller than 15. Peptides with lengths smaller than 15 were arbitrarily treated as coil.

Hydrophobic moment A known phenomenon that causes retention time shifts for isomer peptides is the amphipathicy of the peptides. The amphiphilic helices are those in which one surface of each helix projects mainly hydrophilic side chains, while the opposite surface projects mainly hydrophobic side chains. To quantify the amphiphilicity of a helix, a hydrophobic moment concept proposed by Eisenberg, D.; Weiss, R M.; Terwilliger, T C. The helical hydrophobic moment: a measure of the amphiphilicity of a helix. Nature 1982, 299:371-4, the entire contents of which are incorporated herein by this reference, was used. For an amino acid sequence of N residues and their associated hydrophobicities Hₙ, the mean hydrophobic moment can be calculated from the following definition:

$$〈 {μ}_{H} 〉 = \frac{1}{N} {\{ {[ {\sum }_{n = 1}^{N} {H}_{n} sin ( 2 n π / 3.6 ) ]}^{2} + {[ {\sum }_{n = 1}^{N} {H}_{n} cos ( 2 n π / 3.6 ) ]}^{2} \}}^{1 / 2} \qquad ( 3 )$$

A large value of <μ_{H}> means a large amphipathicy of peptide. The Eisenberg hydrophobicity indices described in Eisenberg, D.; Weiss, R M.; Terwilliger, T C. The hydrophobic moment detects periodicity in protein hydrophobicity. Proc. Natl. Acad. Sci. USA. 1984, 81:140-4, the entire contents of which are incorporated herein by this reference, were used.

ANNs based approaches have advantages in comparison with classical statistical methods that include a capacity to self-learn and to model complex data without the need for detailed understanding of the underlying phenomena.

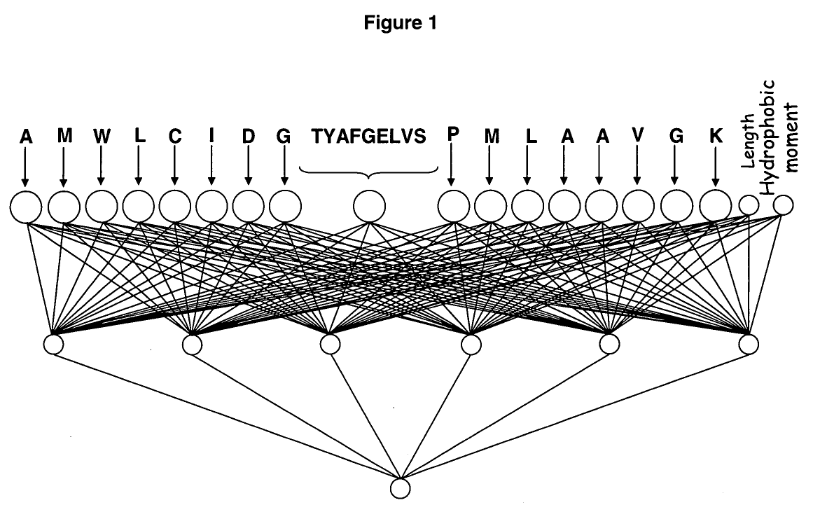

A feed-forward neural network model, sometimes called a backpropagation neural network due to its most common learning algorithm, was used for these experiments. It is composed of large number of neurons, nodes, or processing elements organised into a sequence of layers, as described in Werbos, P. J.; Beyond regression: New tools for predictive and analysis in the behavioural sciences, PhD Thesis, Harvard University, Cambridge, Mass., 1974, and Werbos, P. J.; The Roots of Backpropagation, John Wiley & Sons, New York, 1994, the entire contents of each of which are hereby incorporated herein by this reference. The architecture of these ANN models contain at least two layers: an input layer with one node for each variable in a data vector and, an output layer consisting of one node for each variable to be investigated. Additionally, one or more hidden layers can be added between the input and output layer if the complexity of the data so require. Nodes in any layer can be fully or partially connected to nodes of a succeeding layer as shown in FIG. 1, where each hidden or output node receives signals in parallel. The input signal to a node is modulated by a weight (w) along each link. The net input to a node is thus a function of all signals to a node and all of its associated weights. For example the net input for a node j is given by:

$${net}_{j} = {\sum }_{i} {w}_{ji} {O}_{i} \qquad ( Eq \text{-} 1 )$$

Where i represents nodes in the previous layer, wⱼᵢ is the weight associated with the connection from node i to node j, and Oᵢ is the output of node i.

The final output signal of a node is usually confined to a specified interval, say between zero and one. The net input to the neuron thus underwent an additional transformation using a transfer function. There are several transfer functions available, satisfying a requirement of continuity, set by the backpropagation algorithm. The most popular one is the sigmoid function given by:

$${O}_{j} = \frac{1}{( 1 + {ⅇ}^{- net j} )} \qquad ( Eq \text{-} 2 )$$

In essence, these equations applied to nodes in the hidden and output layers allows these ANNs to perform multiple multivariate non-linear regression using sigmoidal functions, and because of the parallel processing of nodes within each layer, these ANNs have the ability to learn multivariate non-linear functions.

The process of adapting the weights to an optimum set of values is called training the neural network. In order to train the neural network there exist several training algorithms. Examples of such functions are detailed in Rumelhart, D. E.; Hinton, G. E.; Williams, R. J.; Learning internal representations by error propagation, Parallel Distrubuted Processing: Explorations in the Microstructures of Cognition. Vol. 1: Foundations, Rumelhart, D. E.; McClelland, J. L.; (eds.), MIT Press, Cambridge, Mass., USA, pp. 318-362, 1986, the entire contents of which are hereby incorporated herein by this reference. The backpropagation algorithm selected for these experiments is one example, however, the present invention should in no way be viewed as limited to this expample.

In order to enable the comparison of the numerous LC-MS data sets, normalisation of the data was necessary. Two approaches were tested for the normalisation. One uses 5 standard peptides as internal standards and then each run is normalised by using linear regression. The 5 standard peptides used are: 1) ASHLGLAR [SEQ ID No. 1], 2) APRTPGGRR [SEQ ID No. 2], 3) pGlu-P—P-G-G-S—K—V—I-L-F [SEQ ID No. 3], 4) INLKALAALAKKIL [SEQ ID No. 4], 5) FLPLILGKLVKGLL [SEQ ID No. 5]. The second way used the developed predictive capability in order to normalise the different LC runs. In this approach, all the identified peptides are used as internal standards, and their predicted retention time is plotted against the scan number. Linear regression is then used to normalise from run to run. The two methods were compared and proved to be comparable; the second method was used in this study.

1627817 peptides, of which 532448 were different as identified from 5169 LC-MS-MS analyses, were normalised to establish a common timeline so that the same peptides eluted at the same normalized elution time (NET) in the different separations. This optimization scheme of multiple linear regressions normalized the peptide elution times into a common range, between 1 and 0.

In U.S. patent application Ser. No. 10/323,387, filed Dec. 18, 2002, Deinococcus peptides were used for the training set and a fraction of Shewanella peptides were used for testing. In the experiments described herein, peptide identifications from 13 different species were used for the training and testing of this embodiment of the present invention, as shown in table 1.

TABLE 1
Filtering criteria used to determine which peptide identifications
will be selected for the training and testing of the artificial
neural network of one embodiment of the present invention.

| | Charge + 1 with | Charge + 1 with | Charge + 2 | Charge + 3 |
|---|---|---|---|---|
| | MW < 1000 Da | MW > 1000 Da | any MW | any MW |
| Full tryptic | Xcorr > 1.6 | Xcorr > 2.2 | Xcorr > 2.2 | Xcorr > 2.9 |
| Partial Tryptic | None | Xcorr > 2.8 | Xcorr > 3.0 | Xcorr > 3.7 |

In order to keep only peptides for which there was high confidence in the accuracy of the identifications, the peptides were filtered according the criteria shown in table 2. Among the 532448 non-reductant peptides identified by RPLC/ESI-ion-trap MS, 97835 different peptides passed the criteria of table 2. Among them, peptides observed less than 90 times, a total of 96722 peptides, were used as the training set, while peptides observed 90 or more times in different LC-MS runs, for a total of 1113 peptides, were used to test the accuracy of this embodiment of the present invention.

| | Peptides | Peptides | Peptides |
|---|---|---|---|
| Organism/Specie | total | non-reductant | filtered |
| Arabidopsis thaliana | 8510 | 5199 | 1917 |
| Borrelia Burgdorferi | 66066 | 18220 | 7083 |
| Human Cytomegalovirus | 14304 | 6055 | 1688 |
| Deinococcus radiodurans | 586368 | 197477 | 16104 |
| Geobacter Metallireducens | 18307 | 7469 | 3856 |
| Geobacter Sulfurreducens | 154901 | 38026 | 10913 |
| Homo sapiens | 24485 | 11363 | 5455 |
| Rhodobacter sphaerodies | 124341 | 41983 | 11927 |
| Rhodopseudomonas palustris | 12593 | 8174 | 3396 |
| Shewanella oneidensis | 484446 | 154550 | 20363 |
| Synecocystis sp. PCC 6803 | 7282 | 3342 | 2052 |
| Yersinia pestis | 68194 | 26393 | 7491 |
| Saccharomyces cerevisiae | 58020 | 14197 | 5590 |
| Total | 1627817 | 532448 | 97835 |
| Table 2 shows species from which the peptides were identified, reductant and nonreductant number of peptides identified from each | | | |
| # specie, and the number of different peptides used from each specie after filtering with the criteria of table 1. | | | |

These experiments showed improved accuracy of the predictor by incorporating peptide structural information and other analyte descriptors. Table 3 summurises the structural descriptors used in this embodiment, and if they improved the prediction or not. The peptide sequence, the hydrophobic moment and the length increased the accuracy of the prediction after their incorporation. The length didn't improve globaly the accuracy, but it seemed to improve the prediction accuracy of the longer peptides. The other descriptors while normally should affect the peptide retention time, did not improve the prediction accuracy of the ANN model in these experiments. It must be noted, though, that most of these descriptors were prediction themselves, and more accuracate predictions would produce different results.

| Structural descriptors | Improved prediction? |
|---|---|
| Peptide Sequence | Yes |
| Hydrophobic moment | Yes |
| Length | Yes |
| Nearest neighbor | No |
| Hydrophobicity | No |
| Spatial conformation | No |
| (α-Helix, β-sheet, coil) | |

Table 3 showing the peptide descriptors investigated

The sequence of each peptide was defined by using the artificial neural network model. Each amino acid residue position in a peptide could be defined by a 20-dimensional vector. Different configurations were tested in order to see up to which point it was possible to define the peptide sequence and increase the prediction accuracy of the model. Table 4 summarises the results. As shown in the table, for this data set, the best prediction accuracy was obtained when the first 8 and the last 8 amino acid residues of a peptide were defined. This corresponds to a 342 input vectors (320 for the peptide sequence, 20 for the amino acid residues at the middle of the peptide, one for the hydrophobic moment and one for the peptide length. FIG. 1 depicts graphically this ANN architecture. For peptides longer than 16 amino residues, the rest of the amino acid residues were coded as a 20-dimensional vector consisting of the normalized number of each of the 20 amino acid residues making up the amino acid composition of the middle of the peptide. The optimum number of hidden nodes was investigated as well and found that 6 hidden was the optimum number of nodes.

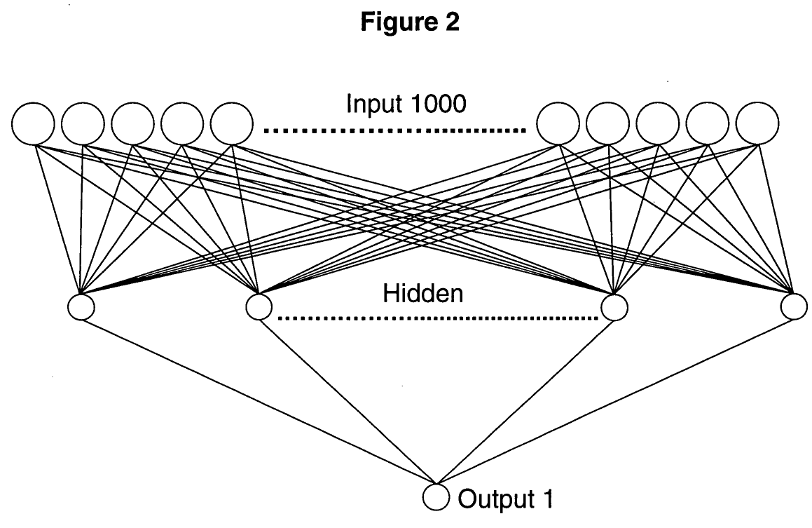

It must be noted here that the only reason that not better accuracies obtained when defining the whole peptide structure is because the training set is not big enough. Ultimately, as shown in FIG. 2, a neural network with 1000 inputs will be optimum to accurately predict the retention time of peptides up to 50 amino acid residues.

| | Input- | | Hydr. | | | TestR- |
|---|---|---|---|---|---|---|
| Lead/end | Vector | Length | Moment | TrainMSE | TestMSE | square |
| ″0/0 | 20 | No | No | 0.0659 | 0.0514 | 0.906 |
| ″0/0 | 21 | Yes | No | 0.0658 | 0.0515 | 0.9059 |
| ″0/0 | 21 | No | Yes | 0.0643 | 0.0492 | 0.9133 |
| ″0/0 | 22 | Yes | Yes | 0.0643 | 0.0492 | 0.9134 |
| ″1/1 | 62 | Yes | Yes | 0.0599 | 0.0454 | 0.9267 |
| ″2/2 | 102 | Yes | Yes | 0.0575 | 0.0412 | 0.9393 |
| ″3/3 | 142 | Yes | Yes | 0.0560 | 0.0391 | 0.9453 |
| ″4/4 | 182 | Yes | Yes | 0.0548 | 0.0369 | 0.9512 |
| ″5/5 | 222 | Yes | Yes | 0.0543 | 0.0353 | 0.9553 |
| ″6/6 | 262 | Yes | Yes | 0.0538 | 0.0349 | 0.9564 |
| ″7/7 | 302 | Yes | Yes | 0.0531 | 0.0343 | 0.9578 |
| ″8/8 | 342 | Yes | Yes | 0.0529 | 0.0334 | 0.9599 |
| ″9/9 | 382 | Yes | Yes | 0.0533 | 0.0337 | 0.9592 |

Table 4 showing the peptide retention time prediction improvement when implementing in the artificial neural network model: sequence information, hydrophobic moment and length of the peptide. The lead/end column refers to the number of amino acid residues defined in the beginning and end of each peptide.

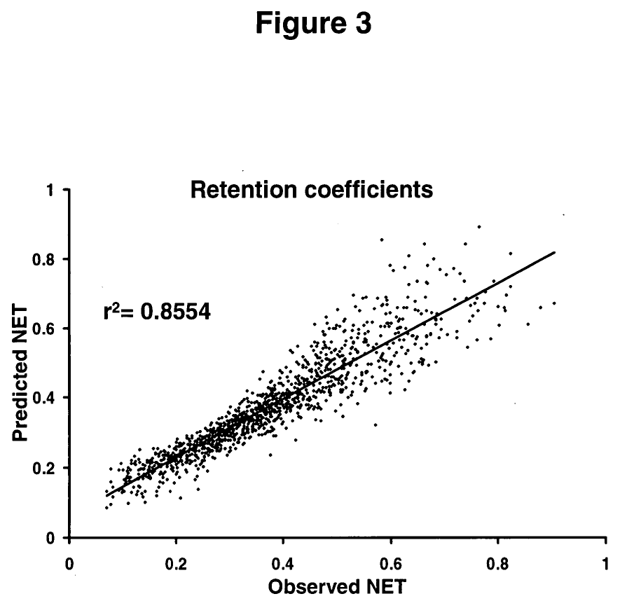

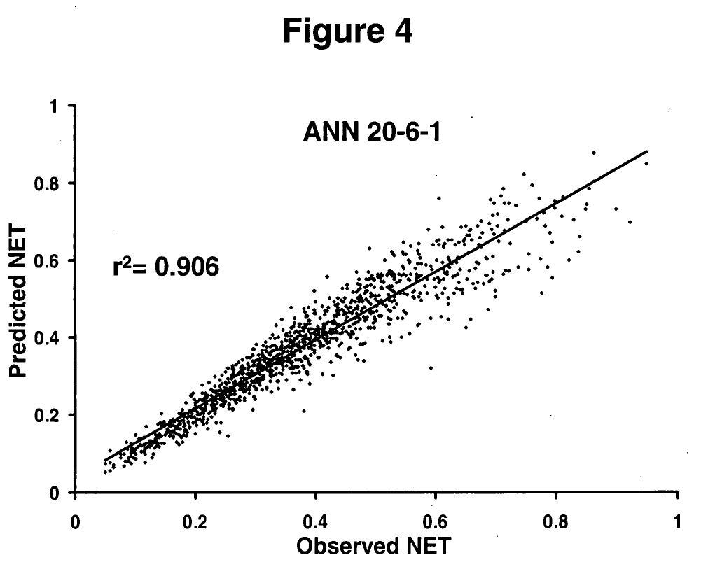

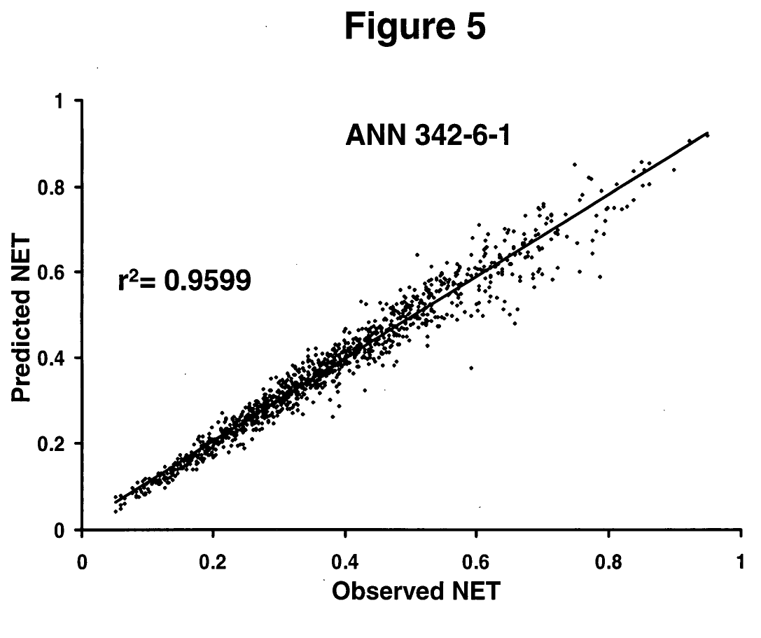

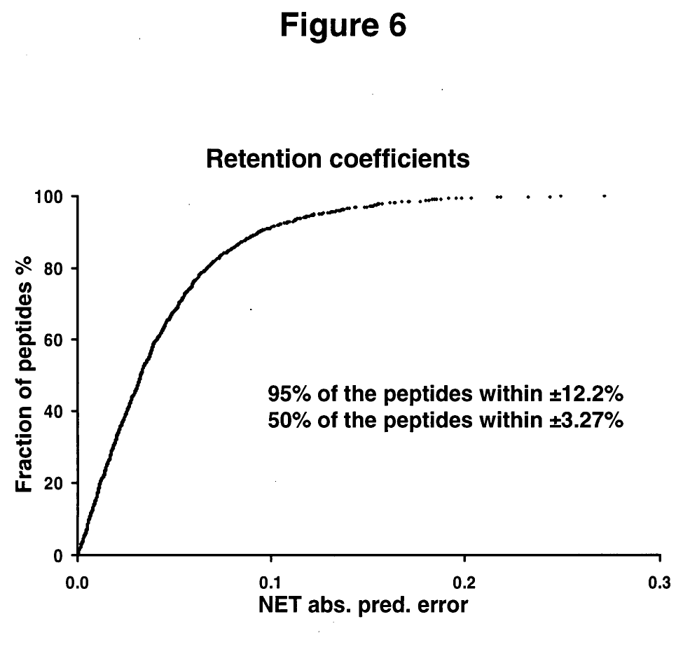

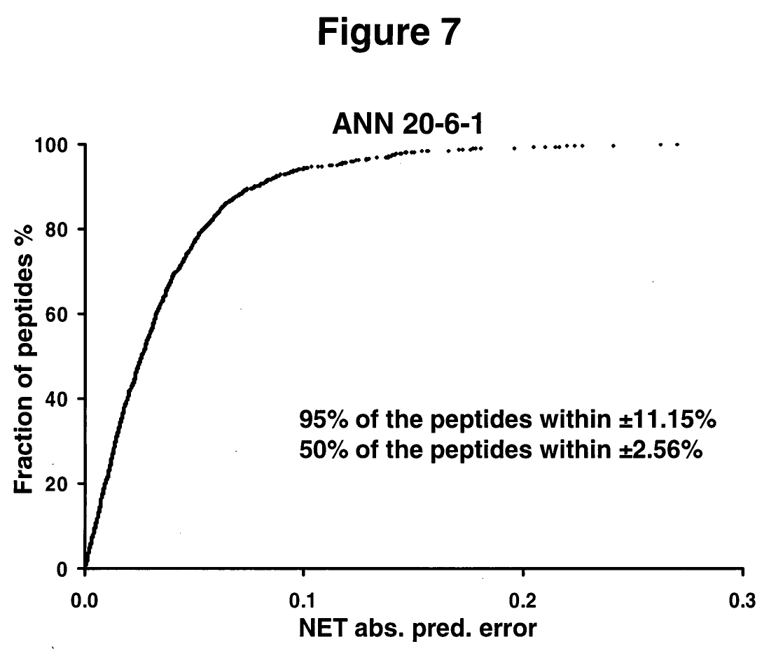

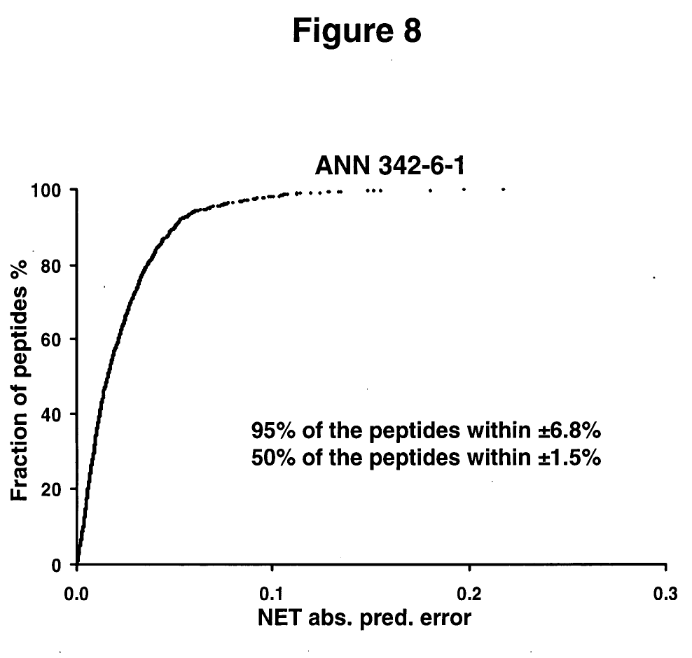

The 342-6-1 ANN architecture was also compared with the 20-6-1 ANN architecture of the prior method and with previous peptide retention time prediction models based on retention coefficients described in Meek, J. L. Proc. Natl. Acad. Sci. U.S.A. 1980, 77, 1632-1636, the entire contents of which are incorporated herein by this reference. The same training and testing data were used for all cases, and FIGS. 3-5 summarise the results. As shown in the Figures, this embodiment of the present invention provides much better predictions with a correlation co-efficient of almost 0.96. FIGS. 6-8 show the normalised elution time prediction error in relation with the % peptide fraction. This embodiment of the present invention is by far better than the prior method which predicted 50% of the peptides within ±6.8% and 95% of the peptides within ±1.5%.

Another advantage of the present invention is that it is able to predict accurately the retention time of isomeric peptides in addition to the isobaric peptides. For example, the isomer peptides LGAGAK (SEQ ID No. 6) (obs. NET=0.12, pred. NET=0.16) and GGLAAK (SEQ ID No. 7) (obs. NET=0.19, pred. NET=0.19) cannot be distinguished with accurate mass measurements, but as they are separated by LC , and the method of the present invention is able to predict accurately their retention time, it is thus possible to distinguish one from the other. All previous models are unable to predict the retention time of such peptides.

### CLOSURE

While a preferred embodiment of the present invention has been shown and described, it will be apparent to those skilled in the art that many changes and modifications may be made without departing from the invention in its broader aspects. The appended claims are therefore intended to cover all such changes and modifications as fall within the true spirit and scope of the invention.

1) a method for predicting the elution time of a chemically related compounds in liquid separations comprising the steps of:
a. providing a data set of known elution times of known peptides,
b. creating a plurality of vectors, each vector having a plurality of dimensions, each dimension representing the position and identity of at least a portion of the amino acids present in each of said known peptides,
c. creating a hypothetical vector by assigning dimensional values for at least one hypothetical peptide, and
d. calculating a predicted elution time for said hypothetical vector by performing at least one multivariate regression fitting said hypothetical peptide to said plurality of vectors.

2) The method of claim 1 wherein said plurality of vectors further comprises vectors having a plurality of dimensions wherein the dimensions of each vector represents the remaining amino acids present in each of said known peptides not represented by said vectors having dimensions representing position and identity.

3) The method of claim 2 wherein said plurality of vectors further comprises vectors describing physical attributes of said peptides.

4) The method of claim 3 wherein said physical attributes are selected from the group consisting of peptide length, nearest neighbor effect, hydrophobic moment, hydrophobicity, peptide mass, molecular volume, quasi sequence order, secondary structure, and combinations thereof.

5) The method of claim 1 wherein said plurality of vectors further comprises vectors describing physical attributes of said peptides.

6) The method of claim 5 wherein said physical attributes are selected from the group consisting of peptide length, nearest neighbor effect, hydrophobic moment, hydrophobicity, peptide mass, molecular volume, quasi sequence order, secondary structure, and combinations thereof.

7) The method of claim 1 comprising the further step of normalizing the known elution times prior to creating said plurality of vectors.

8) The method of claim 1 wherein the multivariate regression is preformed using an artificial neural network.

9) The method of claim 6 wherein the artificial neural network trained with a method selected from the group consisting of gradient descent algorithms and conjugate gradient algorithms.

10) The method of claim 7 wherein the artificial neural network trained with a gradient descent algorithm selected from the group consisting of a backpropagation algorithm and a quickprop algorithm.

11) The method of claim 5 wherein normalization is performed by optimizing a function using multiple regressions.

12) The method of claim 9 wherein the multiple regressions are calculated using a genetic algorithm.

13) The method of claim 9 wherein the function is selected from the group consisting of linear and non-linear functions.

14) The method of claim 1 wherein the liquid separation is performed by a method selected from the group consisting of liquid chromatography, both normal and reverse phase, electrophoretic separations, capillary electrophoresis; field flow fractionation, and combinations thereof.


SEQUENCE LISTING (7 sequences)

SEQ ID NO: 1 - protein, 8 aa, Artificial sequence
  Chain: The artificial sequence was purchased from Sigma, St. Louis, Mo. 2002-2003 Catalogue No. A8651. It is a synthetic peptide, but designed to be the same as Anaphylatoxin C3a fragment 70-77, human.
ASHLGLAR

SEQ ID NO: 2 - protein, 9 aa, Artificial sequence
  Chain: This artificial sequence was purchased from Sigma, St. Louis, Mo. 2002-2003 Catalogue No. A6583. It is believed to be a synthetic construct.
APRTPGGRR

SEQ ID NO: 3 - protein, 11 aa, Hydra Magnipapillata
  misc_feature (1)..(1): pGlu is also known as pyroglutomic acid;
XPPGGSKVILF

SEQ ID NO: 4 - protein, 14 aa, Vespula Lewisii
INLKALAALAKKIL

SEQ ID NO: 5 - protein, 14 aa, Vespa Orientalis
FLPLILGKLVKGLL

SEQ ID NO: 6 - protein, 6 aa, Deinococcus radiodurans
LGAGAK

SEQ ID NO: 7 - protein, 6 aa, Rhodopseudomonas palustris
GGLAAK